(19) United States
(12) Patent Application Publication
(10) Pub. No.: US 2012/0122812 A1
(43) Pub. Date: May 17, 2012

(54) ANTI-AGING AGENT

(76) Inventors: Natalja Nikolaevna Derkach, Kiev (UA); Nykolaj Ivanovich Gumeniuk, Kiev (UA)

(21) Appl. No.: 13/322,458

(22) PCT Filed: May 27, 2010
(86) PCT No.: PCT/UA10/00028

§ 371 Date: Jan. 24, 2012

(30) Foreign Application Priority Data
May 28, 2009 (UA) U200905342
Apr. 16, 2010 (UA) U201004464

(51) Int. Cl. A61K 31/728; A61P 17/00; A61P 19/02

(52) U.S. Cl. 514/54

(57) ABSTRACT

The invention relates to chemical and pharmaceutical industry, in particular, to the medicinal products and medications in the form of intra-articular and intradermal solutions and gels, and can be applied in Orthopedics, Traumatology, Arthrology, Reumatology and Vertebrology as an agent for protection of the cartilages and restoration of fluid balance in the synovial cavity and also in Dermatology, Medical Cosmetology and Plastic Surgery to delay skin ageing.

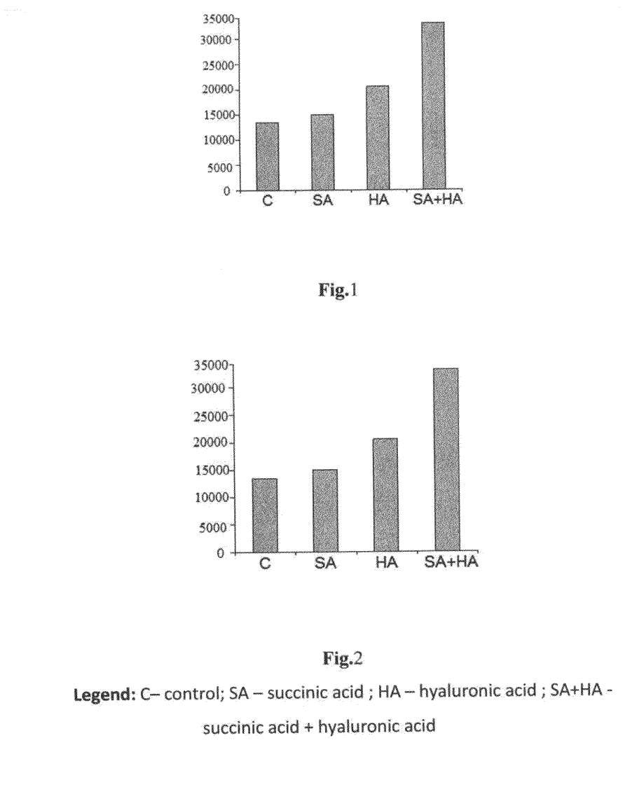

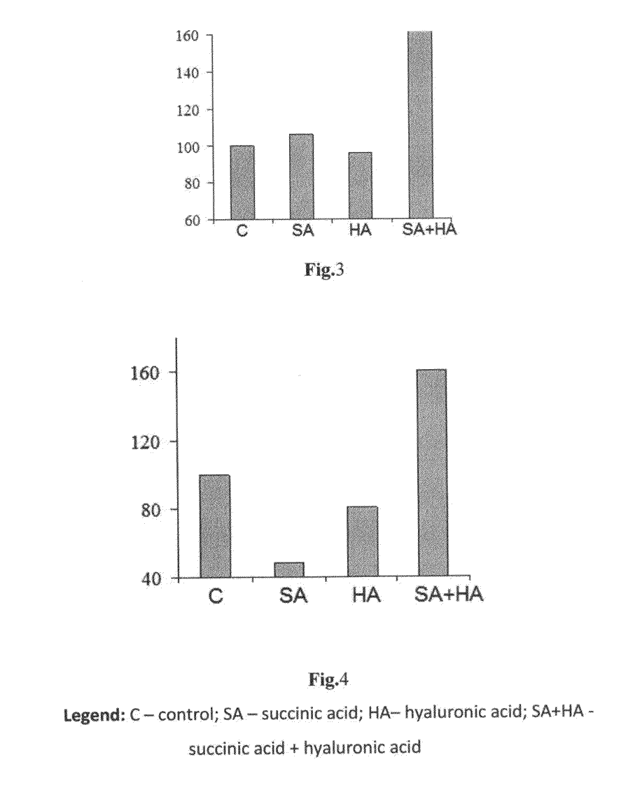

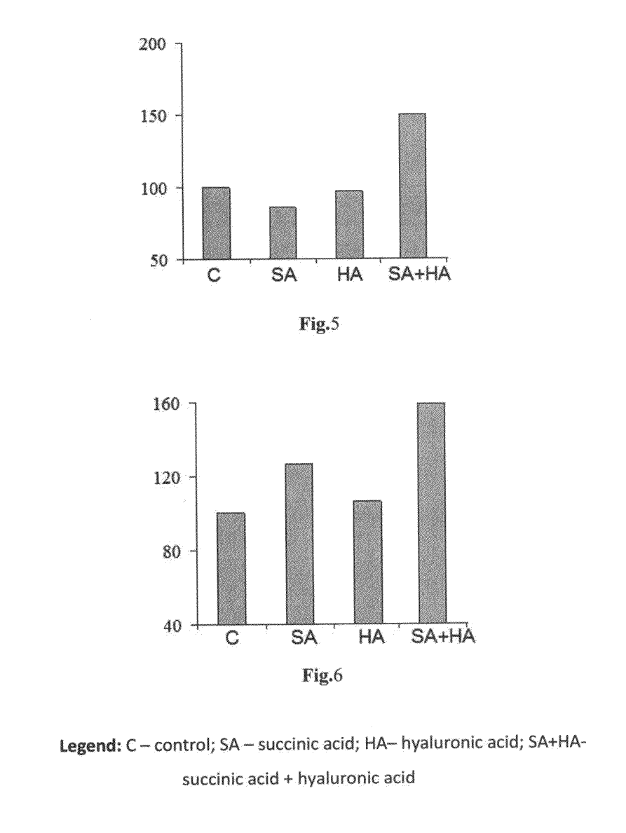

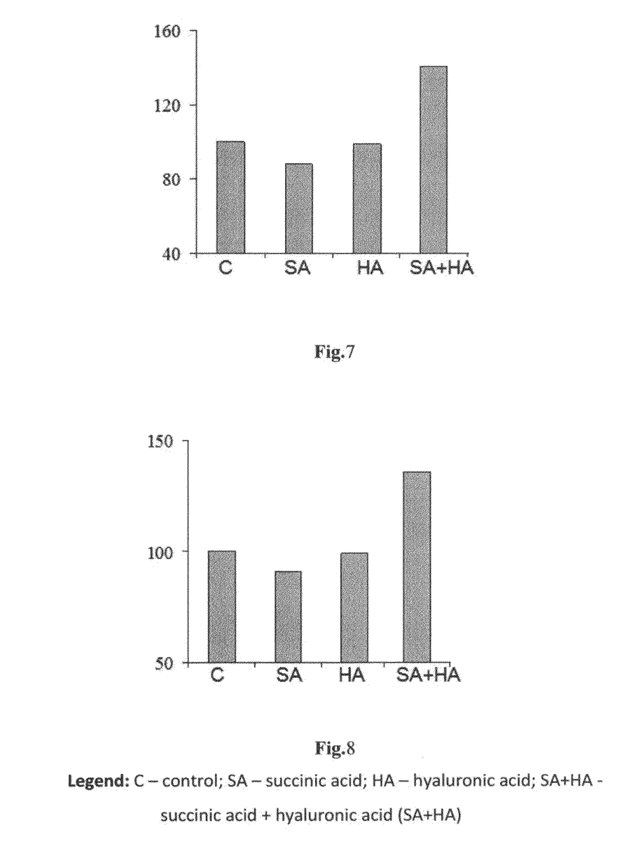

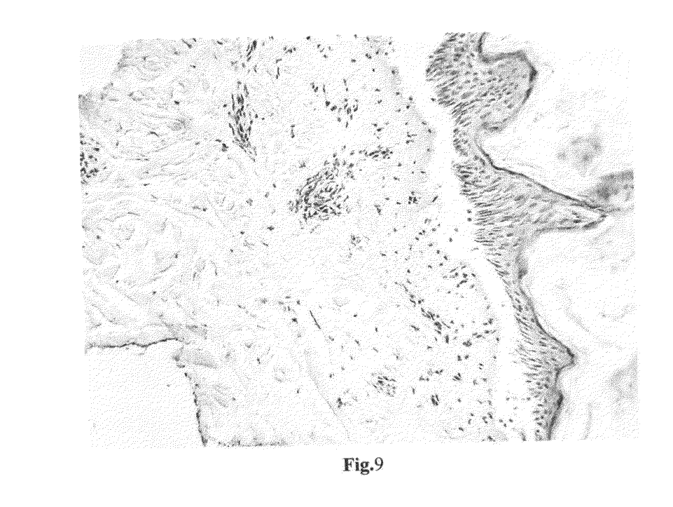

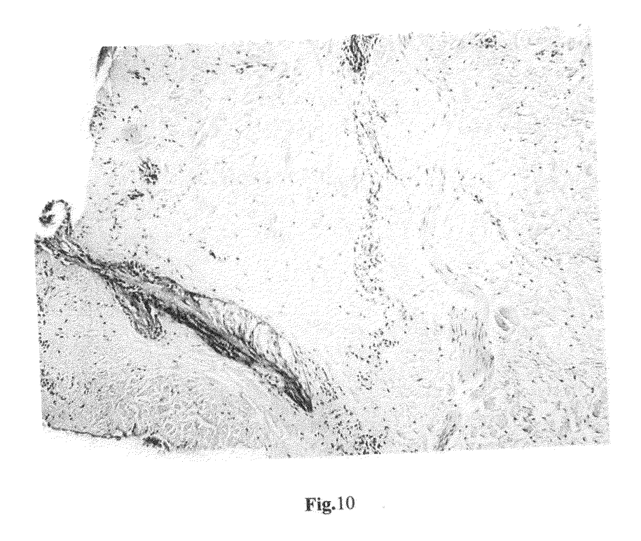

The medicinal agent contains hyaluronic acid, sodium succinate, succinic acid, sodium chloride, sterile water, with following ratio of ingredients, in g/l:

| | |
|---|---|
| hyaluronic acid | 0.5-60.0 |
| sodium succinate | 1.0-40.0 |
| succinic acid | 0.001-10.0 |
| sodium chloride | 1.0-10.0 |
| sterile water | up to 1 litre. |

Complex approach to restoration of the synovial balance within the cavity of the joint was implemented, achieved via the substitutive action of the hyaluronic acid and activation of metabolic processes within the cartilage and a powerful cartilage protection. The above phenomena determine the high clinical efficacy of the medicinal agent, achieved in the treatment of locomotor apparatus.

High efficacy was achieved in terms of cell rejuvenation, restoration of resilience and elasticity of skin, improvement of facial tone, colour and texture, elimination of the symptoms of ageing and fatigue.

The invention relates to chemical and pharmaceutical industry, in particular, to the medicinal products and medications in the form of intra-articular and intradermal solutions and gels, and can be applied in Orthopedics, Traumatology, Arthrology, Reumatology and Vertebrology as an agent for protection of the cartilages and restoration of fluid balance in the synovial cavity and also in Dermatology, Medical Cosmetology and Plastic Surgery to delay skin ageing.

There is a preparation known as Ial-System™ 1.8%, produced by Fidia Company (Italy), applied in restoration and prophylaxis of skin ageing. The above formulation contains only one active substance—hyaluronic acid of non-animal origin. Each package of IAL-System™ includes a pre-filled disposable syringe, containing 1.1 ml. of active solution, not requiring dilution and ready to use. The medication is administered intradermally with thin needles. The preparation is registered in Ukraine—Registration Certificate No. 7551/2008.

The drawback of the above pre-filled syringe formulation is the presence of only one active substance—hyaluronic acid. The latter, being a powerful wetting agent, stimulating endogenous synthesis, cannot exert a multi-factorial influence on the pathogenesis of skin ageing (due to lack of metabolic, restorative and antioxidant effects). In other words, the above mentioned component influences only one link in the pathogenesis of skin ageing, namely, replenishing the water balance.

The preparation known as “Phyto-omolazhivayuschiye,” (Phyto-rejuvenating) has a general tonic, stimulating and restorative effect [Certifications RU 2236859 C2, A61K35/78, 2004]. The above preparation is produced as oral tablets, containing succinic acid 0.0025-0.01 g per tablet.

The preparation has certain shortcomings, discussed below.

The production form of the preparation being tablets, the action upon skin is indirect and lacks potency. Tablets are administered orally, which determines substantially lower bioavailability of active substances compared to injection preparations, since the preparation is subject to substantial gastrointestinal passage. The site of action (derma layer, fibroblasts and amorphic substance) can be penetrated by succinic acid in very low quantities at best, if not at all.

The formulation in question is classified as a food additive, and not as a medication or a medical product, which is yet another confirmation of its weak and indirect influence upon skin

The formulation contains an excessive number of constituents (14 total), some of which are highly allergenic (farina, ginger root, nutmeg), capable of causing a serious anaphylactic reaction.

The intravenous preparation<<Reamberin>>, containing sodium succinate is also known. This infusion preparation is manufactured as infusion vials 200 and 400 ml. each, used as anti-hypoxic and detoxifying agent in acute intoxications of various aetiology.

Reamberin is a balanced isotonic detoxifying infusion solution, containing N-(1-deoxy-D-glucytol-1-il)-N-methylammonium sodium succinate (1.5%), sodium chloride (0.6%), potassium chloride (0.03%), magnesium chloride (0.012%) and sterile water [application instructions can be found at http://www.lib-med.ru/?article=4823; Reamberin is a novel infusion therapy agent, employed in Critical Care. For Application Guidelines—see St. Petersburg Medical Academy of Post-graduate Education; http ://critical.ru/consult/pages/reamberin.htm].

Reamberin is employed only via an intravenous infusion. Lack of intra-articular and intradermal administration options (since the medication is used only intravenously), can become a shortcoming.

Suplasin, an agent used in conditions affecting locomotor apparatus, should be viewed as the most approximate to the invention. Each 1 ml of Suplasin solution contains 10.3 of sodium hyaluronate (which is equivalent to 10.0 mg of hyaluronic acid) and excipients: sodium chloride, sodium phosphate dihydrate, disodium phosphate dodecahydrate, sterile water [by “Bionich Teo” for the Pharmaceutical Company “Vocate S.A.”, Ireland/Greece, http://mozdocs.kiev.ua/likiview.php?id=2582].

Every package od Suplasin contains a disposable syringe, pre-filled with the preparation (0.7 or 2.0 ml), not requiring dilution and ready to use. The agent is administered with needles for intra-articular injections. The medication is registered in Ukraine—Registration Certificate No. UA/2523/01/01.

The disadvantage of the medication above is that the pre-filled syringe contains but one active component—hyaluronic acid. The latter provides a substitutive effect, acting as an implant, which favours restoration of the synovial fluid balance in the cavity of the affected joint, but fails to stimulate cartilage metabolism and cannot exert a comprehensive influence on various pathogenetic links of articular diseases (arthroses, injuries etc.). Preparations containing only hyaluronic acid as a single active substance possess no potential to activate cartilage metabolic processes; aimed at the physiological support of its homeostasis (no strong protection of the cartilages is observed). Besides, the preparation above is used exclusively in locomotor conditions.

The invention is based on the objective to create a more effective medication for restoration of synovial fluid balance in the joint cavity and protection of the cartilages and also for delaying skin ageing by altering the quantitative and qualitative composition of ingredients.

The problem set can be solved by the medicinal agent, containing hyaluronic acid, sodium chloride, sterile water, and, according to the invention, including sodium succinate and succinic acid in the following composition, in g/l:

| | |
|---|---|
| hyaluronic acid | 0.5-60.0 |
| sodium succinate | 1.0-40.0 |
| succinic acid | 0.001-10.0 |
| sodium chloride | 1.0-10.0 |
| sterile water | up to 1 litre. |

The medicinal agent can be used to restore the balance of synovial fluid in the cavity of the joint and protection of the cartilages.

The medicinal agent can be used to delay skin ageing process.

Quantitative ratios of formulation components were found to be within declared values via experiment. It was estimated that the decrease of active ingredient content fails to provide adequate medicinal properties of the preparation, while the increase of content is prone to produce adverse reactions (e.g., increase in pain and/or pseudoallergic reactions).

The medicinal agent applied for review possesses several mechanisms of action due to the presence of hyaluronic acid (sodium hyaluronate), sodium succinate and succinic acid.

Hyaluronic acid (sodium hyaluronate)—is a glycosaminoglycan, composed of D-glucuronic acid and N-acetyl-D-glycosamine disaccharide compounds. It contains not less than 95.0%, but not more than 105.0% of sodium hyaluronate estimated with reference to dried mixture. Hyaluronic acid provides a substitutive effect acting as an implant and imparts optimal physical and chemical properties to synovial fluid, exerts a positive influence on the intercellular substance, subsequently restoring synovial balance in the joints.

Succinic acid (butandionic acid, ethane-1,2-bicarbonic acid—HOOC—CH₂—CH₂—COOH-dibasic carboxylic acid), salts and ethers thereof are referred to as succinates [http://ru.wikipedia.org/wiki].

Succinic acid exerts a stimulating influence on metabolism, stimulates cellular respiration, acts as a powerful energetic by facilitating ATP production, and has a restorative and antioxidant action within the cells. [Reamberin—a novel infusion therapy agent used in Critical Care Medicine. Guidelines.—St. Petersburg Medical Academy of Post-graduate Education; http://critical.ru/consult/pages/reamberin.htm ]

Sodium succinate and succinic acid provide for activation of metabolic processes within the cartilage, therefore ensuring powerful protection of the latter, which determines the high clinical efficiency in the treatment of locomotor conditions. Sodium succinate and succinic acid act upon the cell of the cartilage—the chondrocyte, the key character in natural maintenance of principal physiological processes. The chondrocyte is able to produce “building material” for the joint on its own. Sodium succinate normalizes intracellular tissue metabolism of articular tissues and a number of physiological factors. By acting upon chondrocytes one can achieve restoration of balance within the affected joint in a natural fashion.

Sodium succinate and succinic acid provide for powerful restorative and antioxidant effects, effectively scavenge free radicals, modify tissue metabolism by enhancing cellular respiration, transport of ions, protein synthesis and a vigorous stimulation of energy production (ATP), which determines their high efficiency in cellular rejuvenation, restoration of elasticity and resilience of skin, tone, colour, facial texture, removing signs of ageing and fatigue.

The invention is explained by examples.

### EXAMPLE 1

Composition of the medication:

| | |
|---|---|
| hyaluronic acid | 0.5 |
| sodium succinate | 1.0 |
| succinic acid | 0.001 |
| sodium chloride | 1.0 |
| sterile water | up to 1 litre. |

### EXAMPLE 2

Composition of the medication:

| | |
|---|---|
| hyaluronic acid | 30.25 |
| sodium succinate | 20.5 |
| succinic acid | 5.0 |
| sodium chloride | 5.5 |
| sterile water | up to 1 litre. |

### EXAMPLE 3

Composition of the medication:

| | |
|---|---|
| hyaluronic acid | 60.0 |
| sodium succinate | 40.0 |
| succinic acid | 10.0 |
| sodium chloride | 10.0 |
| sterile water | up to 1 litre. |

The medication is manufactured in the following way.

The following components are used as feed stock:
Hyaluronic acid (sodium hyaluronate) (Pharm. Eur. 4, 01/2002:1472, P. 1915);
Succinic acid (P. 1228 USP 28);
Sodium chloride (State Pharmacopeia of Ukraine: 2004, Appendix 1, P. 422);
Sterile water (State Pharmacopeia of Ukraine: 2004, Appendix 1, P. 307).

The reservoir is charged with the appropriate quantities of sodium chloride, sodium succinate, succinic acid, which are diluted with sterile water to the required volume and mixed thoroughly. The calculated quantity of hyaluronic acid is added to the solution derived; the latter stirred for 2 hours until a homogenous mass is obtained.

Sample splitting and analysis is performed: pH of the solution should be within 5.0-8.5, the quantitative content of the components of the preparation in 1 ml of the prepared solution should correspond to minimal specifications within intervals indicated.

When the solution is certified to comply with the above criteria, it is poured out into syringes with the capacity of 1.0, 1.5, 2.25 or 2.5 ml. and glass vials with the capacity of 6, 8, 10 or 15 ml. The syringes are sealed with rubber plungers. The vials are sealed with rubbers plugs and additionally pressurized with aluminium foil caps. Pre-filled syringes and vials with the solution/gel are sterilized in the autoclave at 105-121° C. (pressure of 1.1 kPa) for 15-60 min. Sterile syringes and vials with the preparation are inspected for mechanical inclusions, labelled and packed. In order to obtain quality certification, quality control is performed for the preparation in accordance to technical specifications for all parameters. Upon the issuance of the quality certificate the finished product is transferred to the storehouse, from which it is distributed to retailers.

The solution (gel) of the complex compound obtained is a transparent colourless liquid (gel). The stability of the solution (gel) was checked under sterilization conditions, also in long-term storage and via accelerated ageing methodology. Control for transparency and discolouration of the solution, as well as the content of active substances is performed before and after sterilization and during the accelerated ageing. It was estimated that the medicinal properties of the claimed medication remain unaltered after sterilization and the subsequent storage for 2 years.

In order to establish the efficacy of the proposed medication, employed to delay skin ageing process, preclinical experimental studies in animals were performed. Toxicity and safety were tested under acute and chronic experimental conditions. The tests performed have proved the medication to be virtually harmless, lacked to demonstrate any allergic reactions and negative influence on animal cell morphology.

The medicinal agent for deceleration of skin ageing process in various concentrations of its constituents was administered via intradermal injections into the dorsum skin of rabbits. There were six animals in each group (active and control). The injection site had been monitored for redness and volume maintenance for 10 days, after which the animals were euthanized on the 2nd, 6th, 8th, 10th, 14th and 21st day, respectively (one animal in a group on each respective day) with histological study of biocompatibility afterwards.

The experimental studies (morphological and histochemical) testified in favour of the proposed agent's ability to optimize cellular function and restore the physiological properties of skin. The medicinal agent for deceleration of skin ageing process in various concentrations of its constituents was proved to possess a longer retention time within the tissues, a substantially better influence upon the structure of derma, collagen and elastin formation, also stronger volume maintenance in comparison with control preparations containing hyaluronic acid only. The difference was particularly visible in the primary stage (71% of initial volume vs. 23% and 21% on the first and second day, respectively). No adverse reactions in experimental animals were detected in microscopy of tissue samples. Morphological studies confirmed the absence of adverse reactions, persistent inflammation, tissue degeneration or necrosis. In administration of the agent presented for review, the phagocytosis of intermediary macrophages was more consistent compared to the preparation containing hyaluronic acid only, which is likely due to a longer interstitial presence.

The medicinal agent for deceleration of skin ageing process is administered intradermally, applying the following techniques.

The papular technique: the preparation is administered as microinjections until small papulae 1-2 mm in diameter are formed 0.5-1 cm apart from each other. The needle is inserted into the upper third of the derma 2-3 mm deep, with its bevel upwards.

The linear technique is used for reinforcement of facial contour and filling of facial wrinkles The needle is inserted parallel to the skin, full length into derma. Injection of the preparation is performed in a retrograde fashion while retracting the needle.

In order to assess the efficacy of the proposed medicinal agent in the treatment of locomotor conditions, clinical and laboratory studies were carried upon patients. The evaluation of the immediate and remote results of applying this medication in patients with osteoarthrosis was based on the criteria below. The intensity of pain syndrome was assessed pre-treatment, during treatment and post-treatment employing the visual 10-grade scale. The integral expression of pain intensity was taken into account (variances at rest and when alert, “starting” pain on palpation, range of motion within joints). The patients were monitored for oedema, contractures and deformities, muscular tensions. Radiography of joints was routinely performed in all patients; MRI and arthroscopy were performed as needed.

The medicinal agent in varying concentrations of respective ingredients was administered via intra-articular injections to patients with osteoarthrosis of major joints of clinical degrees I-III. The age of the patients varied between 28 and 66 years.

As a result of clinical and laboratory studies performed in participating patients, the tolerability of the agent was found to be good. No deterioration of general condition was found; the results of general clinical, laboratory and instrumental studies were as well positive. In 2-3 days after the first intra-articular injection the patients noted alleviation of pain in affected joints both at rest and in physical activity.

A more pronounced effect was observed in patients after the second injection, 8-10 days after the onset of treatment. Marked decrease in the intensity of pain syndrome was accompanied by normalizing of walking patterns, as well as improvement in static and dynamic functions of the affected lower extremities. Rather positive effect persisted for 6 months during the follow-up of patients.

A substantially more pronounced positive impact was found in patients with osteoarthrosis affecting the joints of lower extremities of clinical degrees I-II. The latter manifested mean decrease of overall pain syndrome by 1.6 ±1.4 points after the second injection and by 1.9 ±1.7 points in 6 months, which was significantly better than in controls receiving preparations, containing hyaluronic acid only. Apart from the alleviation of pain, the treatment promoted restoration of muscular trophics, improved range of motion in the affected joints, the patients were able to resume active lifestyles. During clinical supervision no clinical adverse reactions and deteriorations of laboratory indices were detected as a result of administration of the preparation under test.

The data of the clinical and laboratory studies indicated that the medicinal agent is well tolerated by patients with osteoarthrosis of major joints, yielding no negative adverse reactions and alterations of serum parameters. In patients with osteoarthrosis of clinical degrees I, II and III two intra-articular injections of the agent provided for the explicit alleviation of pain syndrome, restoration of both static and dynamic functions of lower extremities and promoted resumption of physical activity.

The ingredients of the medication applied for review exert a targeted influence upon the pathogenesis of locomotive diseases and provide for a substantial pain relief, restoration of static and dynamic functions and resumption of physical activity. The synergistic action of the mentioned components creates an array of physiological effects leading to improvement of physical properties of the cartilage and restoration of the synovial balance.

The components of the medication exert a targeted influence upon the main links of skin ageing pathogenesis, effectively improving skin metabolism, restoring its resilience, elasticity, colour, and texture and providing a lifting effect. The synergy of the aforementioned components produces a complex of physiological effects resulting in restoration of proper skin physiology.

It is only within the expressively claimed contents of constituents and quantitative ratios thereof that the medication exhibits its properties. The formulation as the solution for intra-articular administration preserves its specific activity and stability of the main ingredient in application and storage, as well as the required level of quality, e.g. remains transparent and colourless before and after sterilization and in storage as a solution for prolonged time (in the course of 2 years).

1. The medicinal agent, comprising hyaluronic acid, sodium chloride and sterile water, is different due to the presence of sodium succinate and succinic acid in the following ingredient ratio (in g/l):

| | |
|---|---|
| hyaluronic acid | 0.5-60.0 |
| sodium succinate | 1.0-40.0 |
| succinic acid | 0.001-10.0 |
| sodium chloride | 1.0-10.0 |
| sterile water | up to 1 litre |

2. The application of the medicinal agent according to claim 1 for restoration of synovial balance in joint cavity and cartilage protection.

3. The application of the medicinal agent according to claim 1 for delaying skin ageing process.
